SEQUENCE LISTING (7 sequences)

SEQ ID NO: 1 - protein, 139 aa, Artificial sequence
  Synthetic construct
TPTVAPPAPVYRDHSEKKELPQDEQQDQDHTQEARNQDSDNTQSEHSFEEMYRHILRSQGPFDAVLYYHMMKDEPVVFSTSDGKEYTYPDSLEEEYPPWLTEKEAMNEENRFVTLDGQQFYWPVMNHKNKFMAILQHHQ

SEQ ID NO: 2 - protein, 8 aa, Artificial sequence
  Synthetic construct
SGGSGGSG

SEQ ID NO: 3 - protein, 210 aa, Artificial sequence
  Synthetic construct
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQ

SEQ ID NO: 4 - protein, 201 aa, Artificial sequence
  Synthetic construct
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSD

SEQ ID NO: 5 - protein, 9 aa, Artificial sequence
  Synthetic construct
LYDRLASTV

SEQ ID NO: 6 - protein, 10 aa, Artificial sequence
  Synthetic construct
YFTFDLTALK

SEQ ID NO: 7 - protein, 15 aa, Artificial sequence
  Synthetic construct
PEYMEAIYPVRSNST

(19) United States
(12) Patent Application Publication
(10) Pub. No.: US 2022/0184200 A1
(43) Pub. Date: Jun. 16, 2022

(54) VIRUS-LIKE PARTICLES AND USES THEREOF

(71) Applicant: GEORGIA STATE UNIVERSITY RESEARCH FOUNDATION, INC., Atlanta, GA (US)

(72) Inventors: Christopher F. BASLER, Atlanta, GA (US); JoAnn M. TUFARIELLO, Atlanta, GA (US); Lin WANG, Atlanta, GA (US)

(73) Assignee: GEORGIA STATE UNIVERSITY RESEARCH FOUNDATION, INC., Atlanta, GA (US)

(21) Appl. No.: 17/441,588

(22) PCT Filed: Mar. 20, 2020
(86) PCT No.: PCT/US2020/023911

§ 371 Date: Sep. 21, 2021

Related U.S. Application Data
(60) Provisional application No. 62/821,744, filed on Mar. 21, 2019.

(51) Int. Cl. A61K 39/12; A61K 39/04; A61P 31/06; A61P 31/14; C12N 7/00

(52) U.S. Cl. CPC A61K 39/12; A61K 39/04; A61K 2039/5258; A61P 31/14; C12N 7/00; A61P 31/06

(57) ABSTRACT

Provided herein are virus-like particles and their uses for stimulating anti-pathogenic and anti-cancer immune responses.

### PRIOR RELATED APPLICATIONS

This application claims the benefit of U.S. Provisional Application No. 62/821,744 filed on Mar. 21, 2019, which is hereby incorporated by reference in its entirety.

### STATEMENT REGARDING FEDERALLY FUNDED RESEARCH

This invention was made with government support under AI109664, AI109945, AI114654, AI127009, AI125453, AI109965, and AI127835 awarded by the National Institutes of Health and under HDTRA1-16-1-0033 and HDTRA1-17-1-0005 awarded by the Department of Defense. The government has certain rights in the invention.

### BACKGROUND

Virus-like particles (VLPs) are multi-protein structures that mimic the organization and conformation of authentic native viruses but lack the viral genome, potentially yielding safer and cheaper vaccine candidates. Since VLPs resemble a virus, they can present antigens in their native state to elicit both B and T cell responses. However, despite some advantages, the use of VLPs as vaccines has encountered several difficulties, including inconsistent manufacture of VLPs and relatively low immunogenicity, as compared to live viruses.

### SUMMARY

The present disclosure is based, at least in part, on the discovery of VLPs with enhanced immunogenicity. These VLPs contain a polypeptide adjuvant, i.e., a self-adjuvant, and can be used to stimulate an immune response in a subject, for example, an anti-pathogenic response or an anti-cancer response. The VLPs comprise a surface protein, a matrix protein, a polypeptide that enhances an immune response, and optionally, an intra-VLP protein or a fragment thereof, wherein the polypeptide is a polypeptide adjuvant and wherein the polypeptide is linked to the surface protein, the matrix protein or the intra-VLP protein.

In some embodiments, the surface protein induces a B-cell mediated immune response. In some embodiments, the surface protein is a targeting protein. In some embodiments, the intra-VLP protein or a fragment thereof induces a T cell-mediated immune response.

The polypeptide adjuvant enhances a B-cell mediated immune response, a T cell-mediated immune response, or both a B-cell mediated immune response and a T cell-mediated immune response. In some embodiments, the polypeptide adjuvant comprises one or more signaling domains, such as caspase activation and recruitment domains (CARDs). In some embodiments, the polypeptide adjuvant induces a Type I interferon immune response. In some embodiments, the polypeptide adjuvant comprises a protein or fragment thereof that induces cytokine or chemokine responses, with or without inducing a Type I interferon immune response.

The immune response is optionally an anti-pathogenic immune response. For example, the anti-pathogenic immune response can be an antiviral immune response, an antibacterial immune response, and anti-fungal immune response, or an anti-parasitic immune response. In some embodiments, the immune response is an anticancer immune response

The surface protein optionally comprises a cancer antigen or a bacterial protein. For example, the bacterial protein can be a mycobacterial protein. The bacterial protein can be a bacterial surface protein or a secreted protein. By way of further example, the surface protein can comprise ESAT6 (an exemplary mycobacterial protein), Ag85, or a fragment thereof. Example of VLPs that enhances an anti-bacterial immune response include (a) Ebola virus GP, Ebola virus VP40 and bacterial antigen fused to 2CARD-Ebola NPct and (b) Ebola virus GP, bacterial antigen fused to Ebola virus VP40 and 2CARD-bacterial antigen-Ebola virus NPct.

In some embodiments, the surface protein comprises a viral surface protein, the matrix protein is a viral matrix protein, and/or the intra-VLP protein or a fragment thereof is a viral nucleoprotein (NP) or a fragment thereof. By way of example, the viral NP fragment can be a C-terminal fragment of a viral NP (e.g., a fragment of Ebola virus NP having the amino acid sequence of SEQ ID NO:1).

Optionally, the viral surface protein, the viral matrix protein and the viral NP or a fragment thereof are from the same virus. The virus can be selected from the group consisting of a filovirus, an arenavirus, a paramyxovirus (e.g., a henipavirus), pneumovirus (e.g., respiratory syncytial virus), and an influenza virus.

In some embodiments, the viral surface protein, the viral matrix protein and the viral NP or a fragment thereof are from different viruses. Thus, the viral surface protein, the matrix protein, and the viral NP can be selected from the group consisting of a filovirus, an arenavirus, a paramyxovirus (e.g., a henipavirus), a pneumovirus (e.g., respiratory syncytial virus), and an influenza virus.

The intra-VLP protein or a fragment thereof is optionally linked to a polypeptide comprising two signaling domains (e.g., CARDs). In some embodiments, at least one CARD domain is a CARD domain from a RIG-I-like receptor (e.g., 2 CARD domains from the N-terminus of RIG-I). For example, the RIG-I-like receptor can be retinoic acid-inducible gene-I (RIG-I), Melanoma Differentiation-Associated Protein 5 (MDA5), or laboratory of genetics and physiology 2 (LGP2) (the product of the DHX58 gene). Similarly, a different interferon inducing domain or protein can be used. Examples include the proteins or domains from TIR Domain Containing Adaptor Inducing Interferon-Beta (TRIF) or STING (Stimulator Of Interferon Genes Protein).

In some embodiments, the viral matrix protein is an Ebola virus VP40 matrix protein, the viral surface protein is an Ebola virus glycoprotein (GP) and the viral NP is an Ebola virus NP or a fragment thereof. Optionally, the surface protein and/or the viral NP or a fragment thereof are from different members of the same virus family (e.g., different strains of the filovirus family) or from different viruses (e.g., a filovirus and a non-filovirus virus).

In some embodiments, the viral surface protein is a Lassa virus GPC, the viral matrix protein is a Lassa virus matrix protein Z and/or the viral nucleoprotein is a Lassa virus NP or a fragment thereof.

In some embodiments, the viral surface protein is an influenza virus glycoprotein. In some embodiments, the influenza virus glycoprotein is an influenza virus hemagglutinin (HA), the viral matrix protein is an influenza virus M1 protein and/or the viral nucleoprotein is an influenza virus NP or a fragment thereof.

In some embodiments, the viral surface protein(s) is (are) a henipavirus glycoprotein(s), such as a Nipah virus F protein and/or Nipah virus G protein; the viral matrix protein is a Nipah virus matrix (M) protein; and the viral nucleoprotein is Nipah virus N or a fragment thereof.

In some embodiments, the viral surface protein is a respiratory syncytial virus (RSV) glycoprotein, such as a RSV F protein and/or RSV virus G protein; the viral matrix protein is a RSV matrix (M) protein; and the viral nucleoprotein is RSV N or a fragment thereof.

Also provided is an isolated host cell that expresses any of the VLPs described herein. In some embodiments, the host cell is a mammalian cell or an insect cell.

Further provided is an immunogenic composition comprising any of the VLPs described herein and a pharmaceutically acceptable carrier.

Also provided are methods of making any of the VLPs or immunogenic compositions described herein. The methods include the steps of (a) expressing in a host cell at least one of the VLPs described herein; (b) growing the host cell under conditions that allow the formation of VLPs; and (c) purifying the VLPs. Optionally, the method further comprises preparing the immunogenic composition with the purified VLPs by adding a pharmaceutically acceptable carrier.

In some methods of making VLPs or immunogenic compositions comprising a VLP, the host cell is transfected or infected with one or more recombinant constructs encoding (a) the surface protein; (b) the matrix protein, (c) optionally the intra-VLP protein or a fragment thereof; and (d) a polypeptide that enhances an immune response, wherein the polypeptide is linked to the surface protein, the matrix protein or the intra-VLP protein or a fragment thereof.

Further provided is an immunogenic composition comprising at least one VLP produced by any of the methods provided herein.

Also provided is a method of stimulating an immune response in a subject comprising administering to the subject an effective amount of any of the immunogenic compositions provided herein. The immunogenic composition can be administered to the subject as a single dose or as multiple doses (e.g., initial immunization plus one or more boosts). In some embodiments, the immunogenic composition enhances an immune response in the subject. The immune response can be an anticancer immune response or an immune response against a pathogen. The pathogen can be, for example, a virus, a bacterium, a fungus, or a parasite. The virus, for example, can be selected from the group consisting of a filovirus, an arenavirus, a paramyxovirus (including a henipavirus), a pneumovirus, and an influenza virus. In some embodiments, the virus is selected from the group consisting of Ebola virus, Nipah virus, Lassa virus and influenza virus. In some embodiments, the pathogen is a bacterium, such as a mycobacterium.

### BRIEF DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1A is a diagram illustrating exemplary constructs used to generate various Ebola VLPs (eVLPs). eVLPs were generated by co-expressing a Flag-tagged Ebola virus VP40 matrix protein and Ebola virus glycoprotein (GP). These were co-transfected with empty vector or plasmids that produced hemagglutinin epitope (HA)-tagged full-length Ebola virus nucleoprotein (NP_{FL}), NP C-terminal domain (NP_{CT}), or a region of retinoic acid-inducible gene-I (RIG-I) protein encompassing the two RIG-I caspase activation and recruitment domains (2CARD) fused to NP_{CT} via a Ser-Gly linker (2CARD NP_{CT}).

FIG. 1B shows transmission electron microscopic (EM) images of eVLPs produced with the 2CARD-NP_{CT} construct. The left panel shows the filamentous and curved forms characteristic of Ebola virus (EBOV) virions and eVLPs. The right panel shows immunogold labeling of GP on the eVLP membranes with EM images at higher magnification.

FIG. 1C shows Western blots of purified eVLPs, with blotting for HA-NP constructs, Flag-VP40 and GP. The left panel shows a blot of eVLPs after purification. The right panel shows a blot of purified eVLPs after trypsin-treatment to remove extra-VLP protein.

FIG. 2 shows that the 2CARD-NP_{CT} construct, like a 2CARD without fusion to NP_{CT} (2CARD), induces an interferon (IFN) response. Constructs co-transfected into cells to assess activation of the IFNβ promoter are shown at the top. A full-length RIG-I construct (RIG-I), in which the 2CARD domain is fused to the regulatory helicase domain was included as a control and illustrates that full-length RIG-I does not induce an IFN response in the absence of an activator. 2CARD is the RIG-I signaling domain separated from the regulatory domain, resulting in constitutive signaling activity and expression of the reporter gene. 2CARD-NP_{CT} is the fusion of 2CARD to the NP C-terminal domain (CTD). Empty vector was used as a negative control. IFNβ-luciferase contains the IFNβ promoter upstream of firefly luciferase. Renilla luciferase refers to a reporter plasmid in which Renilla luciferase is constitutively expressed. The lower panel shows relative induction of the IFNβ promoter by the 2CARD and 2CARD-NP_{CT} constructs. Dual luciferase assays were performed 24 hrs post-transfection. Firefly luciferase activity was normalized to Renilla luciferase activity. The data are reported as fold-change of firefly luciferase relative to the empty vector control.

FIG. 3 shows quantitative RT-PCR measurement of expression of IFNβ, IFN-inducible RIG-I and ISG15 and TNF-α mRNA levels following addition of eVLPs containing 2CARD-NP_{CT} to cells after three hours (left column for each VLP), six hours (middle column for each VLP) and twelve hours (right column for each VLP). 2CARD-eVLPS stimulated responses comparable to transfected polyI:C, an activator or RIG-I. The responses required the presence of the 2CARD domain, as eVLPs that contained either NP_{FL} or NP_{CT} without 2CARD did not activate the response. Values were normalized to β actin mRNA levels and reported as fold-change relative to VLPs produced by VP40 alone.

FIG. 4. demonstrates that induction of IFNβ, IFN-inducible RIG-I, ISG56 and IRF-7 as well as cytokine TNF-α mRNA levels depends on the presence of MAVS, a signaling molecule downstream of RIG-I that is required for RIG-I-dependent induction of IFN responses. eVLPs with full-length NP (NP_{FL} VLP), with NP_{CT} or with 2CARD-NP_{CT} were used to infect A549 cells or A549 cells in which MAVS had been deleted. For these experiments, infection with Sendai virus (SeV) served as a positive control activator of IFN signaling. Only the 2CARD-NP_{CT} VLP and SeV induced expression of the measured genes at 4, 8 and 16 hours post-infection in the A549 cells. Induction was abrogated in the MAVS knockout cells, consistent with a model whereby the 2CARD-NP_{CT} VLPs induce IFN responses and TNFα production via the canonical RIG-I pathway.

FIG. 5 shows sustained anti-GP responses of mice at 2, 4, 6 and 8 weeks after a single immunization with 2CARD-NP_{CT} eVLPs. Mice were immunized with the indicated eVLP preparations, bled at the indicated time points and antibody titers assessed by ELISA using recombinant GP as an antigen. PBS injection served as a negative control immunization. 10 μg of eVLP was given to all mice by the intraperitoneal (IP) route, except for the 2CARD-eVLP high dose group which received 25 μg by the IP route.

FIG. 6A provides schematics of the constructs used to make VLPs containing 2CARD and ESAT6, an exemplary Mycobacterial tuberculosis antigen.

FIG. 6B and FIG. 6C show IFN responses induced by VLPs containing both 2CARD and the ESAT6 Mtb antigen. HEK293T cells were transfected with plasmids encoding the following proteins: GP+eVP40; GP+eVP40+2CARD-NP_{CT}; GP+ESAT6-eVP40; GP+2CARD-ESAT6-eVP40; 2CARD-ESAT6-eVP40. After harvesting and purifying the VLPs, the VLPs were added to HEK293T cells. As a control, the HEK293T cells were mock treated. Activation of innate immune responses were determined by measuring levels of mRNAs to interferon β(IFNβ) and the IFNβ-induced gene RIG-I. These mRNA levels were normalized to β-actin mRNA levels and reported as relative copies of the IFNβ (FIG. 6B) or RIG-I mRNAs (FIG. 6C).

FIG. 7 shows that eVLPs produced with a 2CARD-ESAT6-VP40 construct induce an IFN response in a MAVS-dependent manner. Wildtype A549 or MAVS-knockout A549 cells were mock infected or infected with eVLPs of the following types: GP+eVP40, GP+2CARD-NPct, GP+ESAT6-eVP40, GP+2CARD-ESAT6-VP40. Infection was performed by adding eVLPs to the cells. Twelve hours post-Infection, total RNA was isolated from the cells, and quantitative RT-PCR was performed, using oligo(dT) as the RT primer. IFNβ mRNA was quantified and normalized to β-actin mRNA levels.

### DETAILED DESCRIPTION

A handful of VLP-based vaccines are currently commercialized worldwide: GlaxoSmithKline's Engerix (hepatitis B virus) and Cervarix (human papillomavirus), and Merck and Co., Inc.'s Recombivax HB (hepatitis B virus) and Gardasil (human papillomavirus) are some examples. Formed by expression of viral structural proteins, but lacking genetic material that would allow replication, VLPs are safe, in that they cannot cause infection. VLPs are also immunogenic, presenting antigens nearly identical to those presented by authentic virus infection, to elicit both B cell and T cell responses. A limitation of VLPs, including commercially available VLP-based vaccines, is that they often require multiple immunizations and/or administration in combination with adjuvants. Therefore, strategies that augment VLP immunogenicity and/or make co-administration with an adjuvant optional, such as those provided herein, represent a significant advance in vaccine technology.

Ebola virus (EBOV) is a member of the filovirus family of enveloped, negative-sense RNA viruses. The filovirus family is currently divided into 3 genera of which two, Ebolavirus and Marburgvirus, include zoonotic pathogens that cause periodic outbreaks with high fatality rates in humans. Members of the Ebolavirus genus that have caused multiple outbreaks of severe disease include EBOV, Sudan virus (SUDV) and Bundibugyo virus (BDBV). The sole species of the Marburgvirus genus, Marburg virus (MARV), has caused similar lethal outbreaks. The West Africa EBOV epidemic in 2013-2016 resulted in >28,000 human infections and >11,000 deaths, emphasizing the need for effective interventions to prevent and treat filovirus disease. No filovirus vaccine has been licensed for human use. Therefore, there remains a need to continue development of alternate vaccine platforms. Given that the preferred strategy for Ebola virus vaccination is to vaccinate people in the context of ongoing outbreaks, for example, by targeting contacts of patients and where there is imminent risk of exposure to the virus, vaccines that induce rapid immunity would be optimal.

The EBOV matrix protein, VP40, is able to bud from cells to form filamentous membrane-bound particles that resemble authentic EBOV. Co-expression with the viral Ebola virus glycoprotein (GP) results in increased EBOV VLP (eVLP) production with GP present on the surface of the eVLP membrane. Co-expression of the Ebola viral nucleoprotein (NP) with VP40 also enhances budding and results in incorporation of NP into the eVLPs. The combination of VP40, NP and GP yields eVLPs with GP on the surface and VP40 and NP within the eVLP membrane. As described herein, eVLPs that elicit a robust innate immune response, through either the RIG-I or TIR-domain-containing adapter-inducing interferon-β (TRIF)-dependent pathways were made. These eVLPs cause substantially enhanced humoral and cellular immunity. This exemplary approach involved appending the two CARD domains of RIG-I, which constitute a functioning signaling domain, onto EBOV NP C-terminal residues 601-739 (NP_{CT}), a domain that is sufficient for incorporation into eVLPs. When the RIG-I 2CARD domain is fused to NP_{CT} it induces an IFN response, is incorporated into eVLPs, stimulates IFN responses in dendritic cells (DCs) and elicits, after a single immunization in the absence of adjuvant, a robust anti-GP antibody response far greater than the response seen with standard eVLPs. Thus, the VLP platform provided herein is used to enhance the speed and robustness of anti-VLP immune responses against pathogens or cancer antigens.

### VLPs

Provided herein is a VLP comprising a surface protein or fragment thereof a matrix protein or fragment thereof optionally, an intra-VLP protein or a fragment thereof; and a polypeptide that enhances an immune response, wherein the polypeptide is a polypeptide adjuvant, and wherein the polypeptide is linked to the surface protein, the matrix protein or the intra-VLP protein.

As used herein, a virus-like particle or VLP refers to a particle that includes structural proteins, for example, viral structural proteins, but is non-infectious and unable to replicate, as VLPs contain no viral genetic material. VLPs generally include one or more structural proteins, for example, viral structural proteins, including but not limited to capsid, coat, matrix, nucleoprotein, shell, surface and/or envelope proteins, or particle-forming polypeptides derived from these proteins. Optionally, the VLP comprises a surface protein or a particle-forming fragment thereof, a matrix protein or a particle-forming fragment thereof, and a nucleoprotein or a fragment thereof. VLPs can form spontaneously upon recombinant expression of the structural proteins in an appropriate expression system. The VLP as described herein optionally includes a surface protein that is a chimeric protein. For example, a bacterial protein can be fused with the viral surface protein or fragment thereof.

As used throughout, the term optional or optionally means that the subsequently described event or circumstance can or cannot occur and that the description includes instances where the event or circumstance occurs and instances where it does not.

The terms polypeptide, peptide, and protein are used interchangeably herein to refer to a polymer of amino acid residues. As used herein, the terms encompass amino acid chains of any length, including full-length proteins, wherein the amino acid residues are linked by covalent peptide bonds. In the compositions and methods provided herein, a particle-forming polypeptide derived from a particular protein, for example, from a viral or bacterial protein, can be a full-length polypeptide or a fragment thereof. Optionally, the polypeptide derived from a particular protein can be a polypeptide with internal deletions, insertions or substitutions, which has the ability to form VLPs under conditions that favor VLP formation. Accordingly, the polypeptide may comprise the full-length sequence, fragments, truncated and partial sequences, as well as analogs and precursor forms of the reference molecule. The term therefore contemplates deletions, additions and substitutions to the sequence, as long as the polypeptide retains the ability to form a VLP. Thus, the term includes natural variations of the specified polypeptide since variations in proteins often occur between viral or bacterial isolates. The term also includes deletions, additions and substitutions that do not naturally occur in the reference protein, as long as the polypeptide retains the ability to form a VLP. Optionally, substitutions are those which are conservative in nature, i.e., those substitutions that take place within a family of amino acids that are related in their side chains. Such modifications include conservative amino acids substitutions, such that the polypeptide optionally contains one or more conservative amino acid substitutions. The following groups each contain amino acids that are conservative substitutions for one another. These groups are exemplary as other conservative substitutions are known to those of skill in the art.

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

Preferably, the sequences employed to produce VLPs exhibit at least about 50%, 60%, 70%, 80%, 85%, 90%, 95%, or 99% sequence identity to a naturally occurring surface protein, matrix protein or intra-VLP protein. In each case, where specific nucleic acid or polypeptide sequences are recited, embodiments comprising a sequence having at least 80% (e.g. 80%, 85%. 90%, 95%, 99%) identity to the recited sequence are also provided. Identity or similarity with respect to a sequence is defined as the percentage of amino acid residues in the candidate sequence that are identical (i.e., same residue) with the starting amino acid residues, after aligning the sequences and introducing gaps, if necessary, to achieve the maximum percent sequence identity. Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Nucleic acids encoding any of the polypeptide sequences described herein are also provided. The term nucleic acid or nucleotide refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). A nucleic acid sequence encoding a selected polypeptide can include, but is not limited to, cDNA from viral, prokaryotic or eukaryotic mRNA, genomic DNA sequences from viral or prokaryotic DNA, and synthetic DNA sequences. The nucleic acid sequences described herein can be operably linked to each other in any combination. For example, one or more nucleic acid sequences can be expressed from the same promoter and/or from different promoters. As described below, nucleic acid sequences encoding any of the polypeptides described herein can be included on one or more vectors.

As used throughout, a surface protein can be a transmembrane protein or the transmembrane domain of a transmembrane protein. In some embodiments, the surface protein comprises a bacterial protein. The bacterial protein can be a protein or a fragment thereof normally expressed on the surface of a bacterium, a protein or fragment thereof that is normally secreted from a bacterium, a protein or fragment thereof that is normally within a bacterial membrane, or a protein or a fragment thereof that is normally intra-bacterial, for example, normally on the surface of mycobacteria, secreted by a mycobacterium, with a mycobacterial membrane, or within the cytoplasm of a mycobacterium. In some embodiments, the surface protein is a viral surface protein or a chimeric version thereof. For example, the viral surface protein can be a protein or fragment thereof normally expressed on the surface of a virus. In some embodiments, the surface protein is a polypeptide expressed on a cancer cell. For example, the surface protein can be a protein or a fragment thereof normally expressed on the surface of a cancer cell. Alternatively, the surface protein optionally is a viral surface protein fused with a polypeptide expressed by a cancer cell.

Optionally, the surface protein comprises a peptide, for example, an antigen or portion thereof, that induces a B-cell mediated immune response, i.e., an immune response mediated by antibody molecules. The antigen can be a viral antigen, a bacterial antigen, a fungal antigen, a parasitic antigen, or a cancer antigen. The antigen can contain one or more epitopes (either linear, conformational or both) that will stimulate a host's immune-system to make a humoral and/or cellular antigen-specific response. Generally, a B-cell epitope will include at least about 5 amino acids but can be as small as 3-4 amino acids, and can vary in length from about 5 to about 20 amino acids. For example, the epitope can include about 5, 6, 7, 8, 9, 10, 11, 12, 13, 14, 15, 16, 17, 18, 19, or 20 amino acids.

In some embodiments, the surface protein comprises one or more viral B cell epitopes. In some embodiments, the surface protein comprises one or more B cell epitopes from one or more types or strains of a virus. The surface protein optionally comprises one or more bacterial, fungal, or parasitic B cell epitopes. In some embodiments, the surface protein comprises one or more B cell epitopes from one or more types or strains of bacteria, fungus, or parasite. When the surface protein comprises a viral, bacterial, fungal, or parasitic protein, the protein sequences can comprise chimeric polypeptides, for example, an Ebola virus chimeric protein in which all or part (s) of the Ebola virus protein sequence is replaced with sequences from other viruses and/or sequences from other filoviruses strains or other non-filoviruses or replaced with sequences from bacteria, funguses, or parasites. Furthermore, the surface protein is optionally glycosylated.

In some embodiments, the surface protein comprises a peptide that targets the VLP to a particular cell or tissue. The targeting protein or peptide is optionally linked to the surface protein or a fragment thereof. Targeting of VLPs to a particular cell(s) or tissue(s) can also be achieved by linking the targeting peptide to the VLP after VLP assembly or formation. Methods for linking a targeting peptide to a VLP include, but are not limited to, chemical crosslinking (Jegerlehner et al. Vaccine 20: 3104-12 (2002)), click chemistry conjugation (Patel et al. Bioconjugate Chem. 22: 376-87 (2011)), or SpyTag/SpyCatcher reaction (Brune et al. Sci Rep. 6: 19234 (2016))

As used throughout, a matrix protein is a polypeptide or a fragment thereof that promotes assembly of a VLP, optionally, via interaction(s) with the surface protein and/or intra-VLP protein. In some embodiments, the matrix protein is a viral matrix protein or a fragment thereof. Optionally the matrix protein is a chimeric protein. In some embodiments, the matrix protein is fused to a heterologous antigen such as a viral protein or a fragment thereof, a bacterial protein or a fragment thereof, a fungal protein or fragment thereof, or a parasitic protein or fragment thereof. For example, the matrix protein is optionally fused to a T-cell antigen.

As used throughout, an intra-VLP protein or a fragment thereof is a polypeptide that is packaged into any of the VLPs described herein during VLP formation or assembly. An intra-VLP protein can be incorporated into the lumen of the VLP by budding, for example, through direct molecular targeting to the VLP or by means of mass action, wherein the intra-VLP protein is present in the cytosol of the cell and is non-specifically included in the fluid that forms the lumen of the VLP upon VLP budding from the plasma membrane. Although a matrix protein is located inside an VLP, the term intra-VLP protein or fragment thereof, as used herein, is not intended to refer to a matrix protein.

In some embodiments, the intra-VLP protein or fragment thereof interacts with the matrix protein or surface protein to promote assembly of a VLP. Optionally, the intra-VLP protein or a fragment thereof comprises one or more T cell epitopes and induces a T cell-mediated immune response. Generally, a T cell epitope can include at least about 7-9 amino acids. Normally, an epitope will include between about 5 and 20 amino acids, for example, about 5, 6, 7, 8, 9, 10, 11, 12, 13, 14, 15, 16, 17, 18, 19, or 20 amino acids.

As used throughout, a T cell-mediated immune response can include the production of cytokines, chemokines and/or other such molecules produced by activated T cells and/or other white blood cells, including those derived from CD4+ and CD8+ T cells. In some embodiments, the T cell response comprises an antigen-specific response by helper T cells. Helper T cells act to help stimulate the function and to focus the activity of nonspecific effector cells against cells displaying peptide antigens in association with MHC molecules on their surface. T cells can be activated after encountering antigen-presenting cells (APCs) (for example, macrophages, dendritic cells or B cells) that have taken up one or more of the VLPs described herein and present a T cell antigen on their surface via association with the major histocompatibility complex on the APCs. In some embodiments, activating antigen-specific cytotoxic T cells induce apoptosis in cells displaying epitopes of a foreign antigen on their surface. These include, but are not limited to, virus-infected cells, cells with intracellular bacteria, and cancer cells displaying tumor antigens. In some embodiments, the intra-VLP protein is a viral nucleoprotein (NP) or a fragment thereof. The NP or fragment thereof is optionally linked to a T cell antigen. The intra-VLP protein can be a C-terminal fragment of a viral NP, for example, a C-terminal fragment of an Ebola virus NP. In some embodiments the C-terminal fragment corresponds to amino acid residues 601-739 of the Zaire Ebola virus strain Mayinga. By way of example the C-terminal fragment corresponds to (TPTVAPPAPVYRDHSEKKELPQDEQQDQDHTQEARNQDSDNTQSEHSFEEMYRHIL RSQGPFDAVLYYHMMKDEPVVFSTSDGKEYTYPDSLEEEYPPWLTEKEAMNEENRF VTLDGQQFYWPVMNHKNKFMAILQHHQ (SEQ ID NO:1) or a sequence having at least 85%, 90%, 95%, or 99% identity to SEQ ID NO:1.

The VLPs provided herein comprise a polypeptide that enhances an immune response, wherein the polypeptide is a polypeptide adjuvant. The polypeptide adjuvant optionally enhances the immune response as compared to the immune response of a VLP that does not comprise a polypeptide adjuvant, i.e., a non-adjuvanted VLP. In some embodiments, the polypeptide adjuvant enhances the immune response by reducing the number of immunizations necessary to stimulate a lasting immune response, for example, a protective response against a pathogen. In some embodiments, the polypeptide adjuvant is linked to the surface protein, the matrix protein or the intra-VLP protein. Optionally, the polypeptide adjuvant is linked to the surface protein, the matrix protein or the intra-VLP protein via a peptide linker, for example, by a Ser-Gly linker (e.g., SGGSGGSG (SEQ ID NO:2). Additional linkers are described in Klein et al. (2014) Design and characterization of structured protein linkers with differing flexibilities, Protein Eng. Des. Sel. 27(10):325-330, which is incorporated by reference herein in its entirety. Optionally, the peptide linker is at least about 2, 3, 4, 5, 6, 7, 8, 9, 10, 11, 12, 13, 14, 15, 16, 17, 18, 19 or 20 amino acids in length.

By incorporating a polypeptide adjuvant that enhances an immune response, the VLPs provided herein are self-adjuvanted VLPs. By incorporating an adjuvant into the VLPs, the amount of an adjuvant that is not incorporated into the VLP, i.e., a non-VLP adjuvant administered to the subject can be reduced or obviated when the VLPs are administered to a subject. For example, the amount of a non-VLP adjuvant can be reduced by at least 10%, 20%, 30%, 40%, 50%, 60%, 70%, 80%, 90% or 100%. In some embodiments, the self-adjuvanted VLPs described herein reduce the amount of antigen required for stimulating an immune response. It is understood that, although the VLPs provided herein can be administered without a non-VLP adjuvant, the methods provided herein include co-administration of any of the VLPs provided herein with a non-VLP adjuvant. Similarly, the VLP compositions provided herein can comprise the polypeptide adjuvant and optionally further comprise an adjuvant that is packaged into the VLP but is not linked to the matrix protein, the surface protein or the intra-VLP protein non-VLP adjuvant.

The self-adjuvanted VLPs provided herein can be used to enhance an anti-pathogenic immune response (for example, an antiviral, antibacterial, anti-fungal, or anti-parasitic immune response) or to enhance an anti-cancer immune response. In some embodiments, the polypeptide adjuvant enhances a B-cell mediated immune response, a T cell-mediated immune response, or both a B-cell mediated immune response and a T cell-mediated immune response. The polypeptide adjuvant optionally induces a Type I interferon immune response.

In some embodiments, the polypeptide adjuvant comprises one or more signaling domains, such as caspase activation and recruitment domains (CARDs). Optionally, the polypeptide adjuvant comprises at least one CARD domain from a RIG-I-like receptor, such as retinoic acid-inducible gene-I (RIG-I). For example, the polypeptide adjuvant optionally comprises one, two, or more CARDs of RIG-I or MDA5. In some embodiments, the polypeptide adjuvant comprises one or more repeats of the amino acid sequence for RIG-I (MTTEQRRSLQAFQDYIRKTLDPTYILSYMAPWFREEEVQYIQAEKNNKGPMEAATL FLKFLLELQEEGWFRGFLDALDHAGYSGLYEAIESWDFKKIEKLEEYRLLLKRLQPEF KTRIIPTDIISDLSECLINQECEEILQICSTKGMMAGAEKLVECLLRSDKENWPKTLKL ALEKERNKFSELWIVEKGIKDVETEDLEDKMETSDIQ (SEQ ID NO:3)) or one or more repeats of an amino acid sequence having at least 85%, 90%, 95%, or 99% identity with SEQ ID NO:3. In some embodiments, the polypeptide adjuvant comprises one or more repeats of the amino acid sequence for MDA5 (MSNGYSTDENFRYLISCFRARVKMYIQVEPVLDYLTFLPAEVKEQIQRTVATSGNMQ AVELLLSTLEKGVWHLGWTREFVEALRRTGSPLAARYMNPELTDLPSPSFENAHDEY LQLLNLLQPTLVDKLLVRDVLDKCMEEELLTIEDRNRIAAAENNGNESGVRELLKRIV QKENWFSAFLNVLRQTGNNELVQELTGSD (SEQ ID NO:4)) or one or more repeats of an amino acid sequence having at least 85%, 90%, 95%, or 99% identity with SEQ ID NO:4.

In some embodiments, the viral surface protein or a fragment thereof, the viral matrix protein or a fragment thereof and the viral NP or a fragment thereof are from the same virus. The virus can be a virus that infects human or non-human animals. It is understood that the virus can also be the animal counterpart to any human virus described herein. All strains and types and subtypes of each virus described herein are also contemplated. In some embodiments, the viral surface protein or a fragment thereof, the viral matrix protein or a fragment thereof, and the viral nucleoprotein or a fragment thereof are from one or more of a of a filovirus, an arenavirus, a paramyxovirus (e.g., a henipavirus), pneumovirus (e.g., respiratory syncytial virus), and an influenza virus. Additionally, the viral surface protein or a fragment thereof, the viral matrix protein or a fragment thereof, and the viral nucleoprotein or a fragment thereof or their corresponding proteins from one or more of Norovirus; Hepatitis C virus; alphavirus (e.g. eastern equine encephalitis virus, Venezuelan equine encephalitis virus, Western equine encephalitis virus, and Mayaro virus); Bunyavirus (e.g. Rift Valley fever virus, Crimean Congo hemorrhagic fever virus, Severe fever with thrombocytopenia syndrome virus (SFTSV)); and rhabdoviruses (vesicular stomatitis virus, rabies virus, lyssaviruses) could similarly be used. Flaviviruses include, but are not limited to, West Nile virus, Dengue virus (types 1 to 4), yellow fever virus, Japanese encephalitis virus and Zika virus. Arenaviruses include, but are not limited to, lymphocytic choriomeningitis virus, Lujo virus, Lassa fever virus, Machupo virus, Savia virus and Whitewater Arroyo virus. Henipaviruses include, but are not limited to, Hendra virus and Nipah virus. Influenza viruses include, but are not limited to, Influenza virus A, B, C and D. All strains and subtypes of influenza virus are also contemplated, including but not limited to, H1N1, H2N2, H3N2, H7N9, and H5N1 influenza A viruses.

In some embodiments, the viral surface protein is an Ebola virus glycoprotein (GP) or a fragment thereof, the viral matrix protein is Ebola virus VP40 matrix protein or a fragment thereof, and the viral NP is an Ebola NP or a fragment thereof. In some embodiments, the Ebola virus GP, the Ebola virus VP40 matrix protein, or the Ebola virus NP or a fragment thereof is linked to a polypeptide adjuvant comprising one or more signaling domains (e.g., CARDs) as described herein.

The viral surface protein is optionally an influenza virus GP or a fragment thereof, the viral matrix protein is optionally an influenza virus M1 protein or a fragment thereof and the viral NP is optionally an influenza virus NP or a fragment thereof. In some embodiments, the influenza virus GP or a fragment thereof, the influenza virus M1 protein or a fragment thereof or the influenza NP or a fragment thereof is linked to a polypeptide adjuvant comprising one or more signaling domains (e.g., CARDs).

In some embodiments, the viral surface protein is Lassa virus glycoprotein complex (GPC) or a fragment thereof, the viral matrix protein is Lassa virus matrix protein Z or a fragment thereof, and the viral NP is a Lassa virus NP or a fragment thereof. Optionally, the Lassa virus GPC or a fragment thereof, the Lassa virus matrix protein Z or a fragment thereof, or the Lassa virus NP or a fragment thereof is linked to a polypeptide adjuvant comprising one or more signaling domains (e.g., CARDs).

In some embodiments, the viral surface protein is a Nipah virus glycoprotein G or a fragment thereof or a Ninah virus fusion (F) protein or a fragment thereof, the viral matrix protein is a Nipah virus matrix (M) protein or a fragment thereof and the viral NP is a Nipah NP or a fragment thereof. Optionally, the Nipah virus glycoprotein F protein or a fragment thereof, the Nipah virus M protein or a fragment thereof or the Nipah virus NP or a fragment thereof is linked to a polypeptide adjuvant comprising one or more signaling domains (e.g., CARDs).

In some embodiments, the surface protein or a fragment thereof, the viral matrix protein or a fragment thereof, and the viral nucleoprotein or a fragment thereof are from different families of the same virus or from different viruses. For example, and not to be limiting, in some embodiments, the VLP can comprise a viral matrix protein or a fragment thereof from a first virus (for example, an Ebola virus VP40 matrix protein), a surface protein or a fragment thereof from a second virus, and/or in intra-VLP protein or a fragment thereof from a third virus. Optionally, the first, second and third viruses are selected from the group consisting of a Filovirus, an arenavirus, a paramyxovirus (e.g., a henipavirus), a pneumovirus (e.g., respiratory syncytial virus), and an influenza virus. Table 1 shows various combinations.

TABLE 1

| Matrix protein | nucleoprotein | glycoprotein |
|---|---|---|
| VP40 | Ebola virus NP | Ebola virus GP |
| VP40 | other filovirus NP | other filovirus GP |
| VP40 | Ebola virus NP | Arenavirus GP |
| VP40 | Ebola virus NP | paramyxovirus (including |
| | | henipaviruses) glycoproteins |
| VP40 | Ebola virus NP | pneumovirus (including |
| | | respiratory syncytial virus) |
| | | glycoproteins |
| VP40 | Ebola virus NP | influenza virus |
| | | hemagglutinin and or |
| | | neuraminidase |
| VP40 | Ebola virus NP | chimeric Ebola virus/other |
| | | virus glycoprotein to |
| | | enhance incorporation into |
| | | VLPs |
| Nipah virus | Nipah virus | Nipah virus fusion (F) |
| matrix protein | nucleoprotein | and/or glycoprotein (G) |
| (M) | | |
| Respiratory | Respiratory syncytial | Respiratory syncytial virus |
| syncytial virus | virus nucleoprotein | fusion and/or glycoprotein |
| matrix protein | | |
| Other | Other paramyxovirus | Other paramyxovirus fusion |
| paramyxovirus | nucleoprotein | and/or other paramyxovirus |
| matrix (M) | | glycoprotein |
| Lassa virus Z | Lassa virus | Lassa virus glycoprotein |
| protein | nucleoprotein | |
| Other arenavirus | Other arenavirus | Other arenavirus |
| Z protein | nucleoprotein | glycoprotein |

The VLP can comprise a surface protein (e.g., a viral protein, a bacterial protein, a fungal protein, a parasitic protein, or a fragment thereof), a viral matrix protein (for example, an Ebola virus VP40 matrix protein), and a viral intra-VLP protein (for example, an Ebola virus NP) or a fragment thereof. In some embodiments, the surface protein or a fragment thereof, the viral matrix protein or a fragment thereof, or the viral intra-VLP protein or a fragment thereof is linked to a polypeptide adjuvant comprising one or more signaling domains (e.g., CARDs).

Optionally, the surface protein is a cancer antigen, the matrix protein is a viral matrix protein (for example, Ebola virus VP 40 matrix protein) or a fragment thereof and the intra-VLP protein is a viral nucleoprotein (for example, Ebola virus NP) or a fragment thereof. In some embodiments, the cancer antigen, the viral matrix protein or a fragment thereof, or the viral intra-VLP protein or a fragment thereof is linked to a polypeptide adjuvant comprising one or more CARDs.

Optionally, the VLP directed to a bacterial target comprises a bacterial protein or a fragment thereof, a matrix protein or a fragment thereof, and an intra-VLP protein or a fragment thereof. The VLP can comprise a viral surface protein; 2CARDs, optionally fused to a matrix protein or an intra-VLP protein; and a bacterial protein, optionally fused to a matrix or intra-VLP protein.

### Expression Vectors and Host Cells

Sequences encoding the desired polypeptides to be incorporated into any of the VLPs described herein can be cloned into any suitable vector or replicon for expression. Numerous cloning vectors are known to those of skill in the art, and one of skill in the art can readily select appropriate vectors and control elements for any given host cell type in view of the present specification as well as information known in the art about expression (see Ausubel et al. Current Protocols in Molecular Biology, Wiley & Sons; and Green, 1988; and Sambrook Molecular Cloning—A Laboratory Manual, 4th Ed., Cold Spring Harbor Laboratory Press, New York (2001)).

Non-limiting examples of vectors that can be used to express sequences that assemble into VLPs include viral-based vectors (for example, retrovirus, adenovirus, adeno-associated virus, lentivirus), baculovirus vectors, plasmid vectors, non-viral vectors, mammalian vectors, mammalian artificial chromosomes, and combinations thereof.

Host cells can be transformed, transfected, or infected with one or more expression vectors containing nucleic acid encoding the polypeptides, under conditions and for an amount of time sufficient to allow expression of the proteins and formation of VLPs. Such conditions for protein expression vary with the choice of the expression vector and the host cell and are ascertainable by one skilled in the art through routine experimentation.

Expression vectors typically contain coding sequences and expression control elements that allow expression of the coding regions in a suitable host. For example, sequence(s) encoding the polypeptide(s) can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. In addition, the expression vector can include more than one replication system, such that it can be maintained in two different organisms, for example, in mammalian or insect cells for expression and in a prokaryotic host for cloning and amplification.

Typical promoters used for mammalian cell expression include the SV40 early promoter, a CMV promoter (such as the CMV immediate early promoter, optionally including intron A), RSV, HIV-LTR, the mouse mammary tumor virus LTR promoter (MMLV-LTR), FIV-LTR, the adenovirus major late promoter (Ad MLP), and the herpes simplex virus promoter, among others. Other nonviral promoters, such as a murine metallothionein promoter can also be used for mammalian expression. Enhancer elements can also be used to increase expression levels of the constructs, for example, in mammalian host cells. Examples include the SV40 early gene enhancer, the enhancer/promoter derived from the long terminal repeat (LTR) of the Rous Sarcoma Virus, and elements derived from human CMV, to name a few.

It is understood that an expression vector may contain one or more nucleic acid sequences as described herein. For example, in some embodiments, a single vector comprises sequences encoding all of the proteins found in a VLP. Alternatively, multiple vectors may be used (for example, multiple constructs, each encoding a single polypeptide-encoding sequence or multiple constructs, each encoding one or more polypeptide-encoding sequences). In embodiments in which a single vector comprises multiple polypeptide-encoding sequences, the sequences may be operably linked to the same or different transcriptional control elements within the same vector.

The ability of a surface protein, a matrix protein, and an intra-VLP protein or fragments thereof to self-assemble into VLPs with antigenic proteins presented on the surface allows these VLPs to be produced in any host cell by the co-introduction of the nucleic acid sequences encoding the surface protein, matrix protein, and the intra-VLP protein or fragments thereof. In some embodiments, the sequence(s) are stably integrated into a host cell. In some embodiments, the sequence(s) are transiently integrated into a host cell. Suitable host cells include, but are not limited to, bacterial, yeast, insect, Xenopus, avian, mammalian, and plant cells. Host cells that comprise or secrete any of the VLPs described herein are also provided. Host cells that express sequences described herein to produce self-assembly of VLPs are also provided herein. Populations of any of the host cells described herein are also provided.

### Methods of Making VLPs

Provided herein is a method of making any of the VLPs described herein. The method comprises the steps of (a) expressing in a host cell at least one VLP described herein; (b) growing the host cell under conditions which allow the formation of VLPs; and (c) purifying the VLPs. Also provided is a method of making an immunogenic composition comprising any of the VLPs described herein and a pharmaceutically acceptable carrier. The method comprises (a) expressing in a host cell at least one VLP described herein; (b) growing the host cell under conditions which allow the formation of VLPs; (c) purifying the VLPs; and (d) preparing the immunogenic composition with the purified VLPs (e.g., by adding a pharmaceutically acceptable carrier). An immunogenic composition comprising at least one VLP produced by any of the methods described herein is also provided.

In some embodiments, the host cell is transfected or infected with one or more recombinant constructs encoding (a) the surface protein; (b) the matrix protein, (c) the intra-VLP protein or fragments thereof; and (d) a polypeptide that enhances an immune response, wherein the polypeptide is linked to the surface protein, the matrix protein or the intra-VLP protein or fragments thereof when expressed in the host cell.

When expression vectors containing the nucleic acid sequences encoding the proteins necessary for VLP formation are introduced into host cell(s) and subsequently expressed, the VLPs assemble and are then released from the cell surface into the culture media. Depending on the expression system and host selected, the VLPs are produced by growing host cells transformed by an expression vector(s) under conditions whereby the particle-forming polypeptides are expressed and VLPs can be formed. The selection of the appropriate growth conditions is within the skill of the art. If the VLPs accumulate intracellularly, the cells can be disrupted, using chemical, physical or mechanical means, which lyse the cells yet keep the VLPs substantially intact. Such methods are known to those of skill in the art. Alternatively, VLPs may be secreted and harvested from the surrounding culture media. See, for example, Kim and Kim, Letters in Applied Microbiology 64: 111-123 (2016)). The particles are then isolated (or substantially purified) using methods that preserve the integrity thereof, such as, by density gradient centrifugation, e.g., sucrose gradients, PEG-precipitation, pelleting, and the like (see, for example, Vicente et al. J. Invertebr. Pathol. 107: S42-8 (2011); and Peyret Journal of Virological Methods 225: 59-63 (2015)), as well as standard purification techniques including, but not limited to, ion exchange and gel filtration chromatography.

### Immunogenic Compositions

Any of the VLPs described herein or mixtures thereof can be provided in an immunogenic composition, for example, a vaccine. These include for example, an immunogenic composition comprising an effective amount of any of the VLPs described herein or mixtures thereof and a pharmaceutical carrier. The term carrier means a compound, composition, substance, or structure that, when in combination with the VLP(s), aids or facilitates preparation, storage, administration, delivery, effectiveness, selectivity, or any other feature of the VLP(s) for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, dextrose, proteins, polysaccharides, polylactic acids, polyglycolic acids, polymeric amino acids, amino acid copolymers, lipid aggregates (for example, oil droplets or liposomes) and water. Typically, the carrier is a molecule that does not itself induce the production of antibodies harmful to the subject receiving the composition.

In some embodiments, the immunogenic composition comprises an adjuvant that is not incorporated into the VLP. These include, but are not limited to aluminum salts (for example, aluminum hydroxide, aluminum phosphate, aluminum sulfate, etc.); muramyl peptides; bacterial cell wall components; Complete Freunds Adjuvant (CFA); Incomplete Freunds Adjuvant (IFA); cytokines (for example, interleukins (IL-1, IL-2, etc.), macrophage colony stimulating factor (M-CSF), tumor necrosis factor (TNF), beta chemokines (MIP, 1-alpha, 1-beta Rantes, etc.); and detoxified mutants of cholera toxin (CT), a pertussis toxin (PT), or an E. coli heat-labile toxin (LT), to name a few.

Immunogenic compositions comprising any of the VLPs described herein can be prepared according to standard techniques and further comprise a pharmaceutically acceptable carrier. These compositions are usually sterile. The pharmaceutical compositions can also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Lloyd V. Allen et al, editors, Pharmaceutical Press (2012).

Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like (for example, sodium acetate, sodium lactate, sodium chloride, potassium chloride, and calcium chloride). Any lipid containing suspension may include lipid-protective agents that protect lipids against free-radical and lipid-peroxidative damages on storage. Lipophilic free-radical quenchers, such as alpha tocopherol and water-soluble iron-specific chelators, such as ferrioxamine, are suitable.

The concentration of the VLPs in the immunogenic compositions can vary widely, i.e., from about 0.05% to about 30% by weight and will be selected primarily by fluid volumes and viscosities, in accordance with the particular mode of administration selected. Alternatively, the VLPs can be dried or lyophilized and resuspended to a desired concentration in water or buffers at the time of use.

### Administration

Provided herein is a method of stimulating an immune response in a subject comprising administering to the subject an effective amount of any of the VLPs or mixtures of various VLPs described herein. Also provided is a method of stimulating an immune response in a subject comprising administering to the subject an effective amount of any of the immunogenic compositions comprising any of the VLPs described herein or a mixture of VLPs described herein. As used throughout, immunogenic refers to an agent that stimulates the immune system of a living organism, so that one or more functions of the immune system are increased and directed towards the immunogenic agent.

In some embodiments, a population of VLPs that stimulate an immune response against a first antigen can be co-administered with a population of VLPs that stimulate an immune response against a second antigen or a second epitope or a second epitope on the first antigen. For example, a population of VLPs that stimulate an immune response against a first cancer antigen can be co-administered with a population of VLPs that stimulate an immune response against a second cancer antigen. For example, a population of VLPs that stimulate an immune response against a first viral antigen can be co-administered with a population of VLPs that stimulate an immune response against a second viral antigen. For example, a first population of VLPs that contain an antigen, but lack the 2CARD domain, can be administered with a second population of VLPs that contain a 2CARD domain, but lack the antigen. It is understood that the methods are not limited to stimulating immune responses against a first and second antigen, as several populations of VLPs that stimulate an immune response against a first, second, third, fourth, fifth antigen etc., respectively, can be co-administered to the subject.

Also provided is a method of preventing or reducing the risk of pathogenic infection in a subject comprising administering to the subject an effective amount of any of the VLPs or VLP-containing immunogenic compositions or mixtures thereof described herein. When the terms prevent, preventing, and prevention are used herein in connection with a given treatment for a given condition (e.g., preventing pathogenic infection), they mean that the treated subject either does not develop a clinically observable level of the condition at all, or develops it more slowly and/or to a lesser degree than the subject would have absent the treatment. These terms are not limited solely to a situation in which the subject experiences no aspect of the condition whatsoever. For example, a treatment will be said to have prevented the condition if it is given during exposure of a subject to a stimulus that would have been expected to produce a given manifestation of the condition, and results in the subject's experiencing fewer and/or milder symptoms of the condition than otherwise expected. A treatment can prevent infection by resulting in the subject's displaying only mild overt symptoms of the infection; it does not imply that there must have been no penetration of any cell by the infecting microorganism.

Similarly, reduce, reducing, and reduction as used herein in connection with the risk of infection with a given treatment (e.g., reducing the risk of pathogenic infection) refers to a subject developing an infection more slowly or to a lesser degree as compared to a control or basal level of developing an infection in the absence of a treatment (e.g., administration of VLPs or a VLP-containing immunogenic composition). A reduction in the risk of infection may result in the subject's displaying only mild overt symptoms of the infection or delayed symptoms of infection

As used throughout, by subject is meant an individual. The subject can be an adult subject or a pediatric subject. Pediatric subjects include subjects ranging in age from birth to eighteen years of age. Thus, pediatric subjects of less than about 10 years of age, five years of age, two years of age, one year of age, six months of age, three months of age, one month of age, one week of age, or one day of age are also included as subjects. Preferably, the subject is an animal, for example, a mammal such as a primate, and, more preferably, a human. Non-human primates are subjects as well. The term subject includes domesticated animals (such as cats, dogs, etc.), livestock (for example, cattle, horses, pigs, sheep, goats, etc.), poultry (e.g., chickens, turkeys, etc.), fish (e.g., farm-raised fish), and laboratory animals (for example, ferret, chinchilla, mouse, rabbit, rat, gerbil, guinea pig, etc.). Thus, veterinary uses and medical formulations are contemplated herein.

The VLPs or VLP-containing immunogenic compositions are administered in a number of ways, including, for example, orally, intranasally, via inhalation, via nebulizer, parenterally, intravenously, intraperitoneally, intracranially, intraspinally, intrathecally, intraventricularly, intramuscularly, intratumorally or near a tumor, subcutaneously, intracavity, or transdermally. Effective doses for any of the administration methods described herein can be extrapolated from dose-response curves derived from in vitro or animal model test systems.

The VLPs or VLP-containing immunogenic compositions can be administered prior to, concurrent with, or subsequent to delivery of other VLPs or immunogenic compositions, for example, a vaccine. Also, the site of VLP administration may be the same or different as other vaccine compositions that are being administered. The VLPs or VLP-containing immunogenic compositions can be administered as a single dose schedule or as multiple doses. In some embodiments, the VLPs or the VLP-containing immunogenic compositions induce an immune response, for example, a protective immune response, with fewer doses as compared to a non-self-adjuvanted VLP delivered with an adjuvant or a non-VLP based immunogenic composition delivered with an adjuvant. In some embodiments, the VLPs or the VLP-containing immunogenic compositions induce an immune response with a single dose. In some embodiments, the VLPs or the VLP-containing immunogenic composition induces a protective immune response with a single dose. As set forth above, an immune response can be a humoral immune response and/or cellular immune response leading to the activation or proliferation of B- and/or T-lymphocytes and/or antigen presenting cells. In some embodiments, the immune response is an immune response against a pathogen in the subject, for example, a virus or a bacterium. Examples of viruses include a Filovirus, an arenavirus, a paramyxovirus (e.g., a henipavirus), pneumovirus (e.g., respiratory syncytial virus), and an influenza virus in the subject. More specifically, the virus can be an Ebola virus, Nipah virus, Lassa virus or influenza virus in the subject. In some embodiments, the immune response is an immune response against a bacterium. Examples of bacteria include mycobacteria, for example, M. tuberculosis.

In some embodiments, the immune response is against a cancer antigen. Examples of cancer or tumor antigens include, but are not limited to non-mutated shared antigens (e.g., MAGE, BAGE, RAGE, and NY-ESO), that are expressed in testes and in multiple tumor cells; differentiation antigens (e.g., prostate-specific membrane antigen (PSMA) and prostate-specific antigen (PSA) in prostate carcinoma, Mart1/MelanA and tyrosinase present in many melanoma, and carcino embryonic antigen (CEA) present in a large percentage of colon cancers) that are tissue restricted and present in lineage-specific tumor cell; mutated oncogenes and tumor suppressor genes (e.g., mutated ras, rearranged bcr/abl, mutated p 53); oncovirus-derived epitopes (e.g., the human papillomavirus-encoded E6 and E7 proteins, Epstein-Barr virus (EBV)-associated antigens present in primary brain lymphoma); and nonmutated oncofetal proteins such as CEA, α-fetoprotein, and survivin. In some embodiments, a cancer antigen expressed by the subject, for example, an antigen expressed on the surface of a tumor cell in a subject is identified. After identification of the tumor antigen, VLPs comprising a surface protein that includes the tumor antigen or an intra-VLP protein comprising a tumor antigen can be made and administered to the subject according to the methods described herein.

The dosage regimen may be selected in accordance with a variety of factors including type, species, age, weight, sex, medical condition of the patient; the severity of the condition to be treated; the route of administration; the renal and hepatic function of the patient; and the particular pharmaceutical composition employed. One of ordinary skill in the art can readily determine and prescribe the effective amount of the composition (and potentially other agents including therapeutic agents).

Ranges may be expressed herein as from about one particular value, and/or to about another particular value. When such a range is expressed, another aspect includes from the one particular value and/or to the other particular value. Similarly, when values are expressed as approximations, by use of the antecedent about, it will be understood that the particular value forms another aspect. It will be further understood that the endpoints of each of the ranges are significant both in relation to the other endpoint, and independently of the other endpoint.

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### Examples

### VLPs

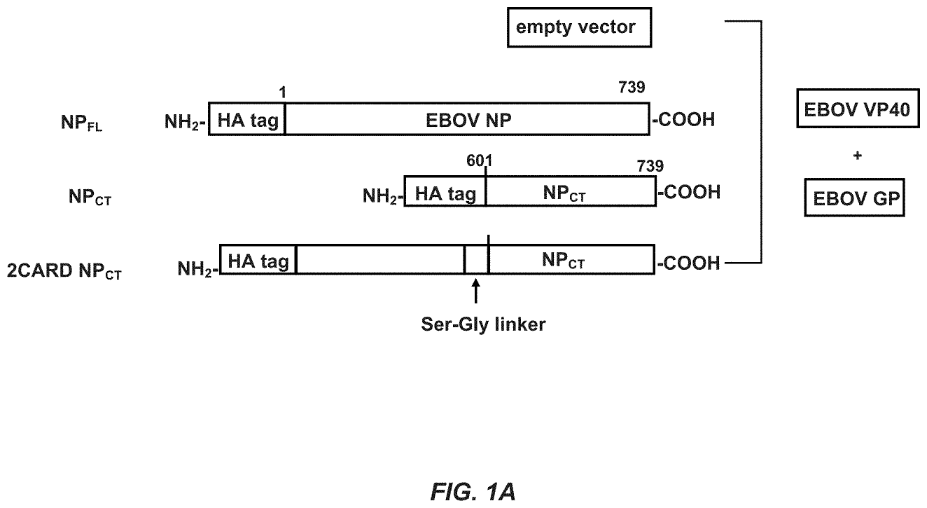

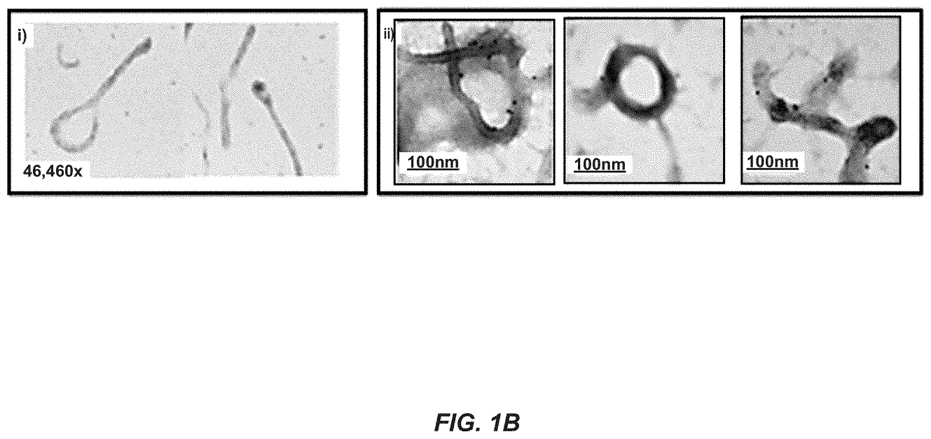

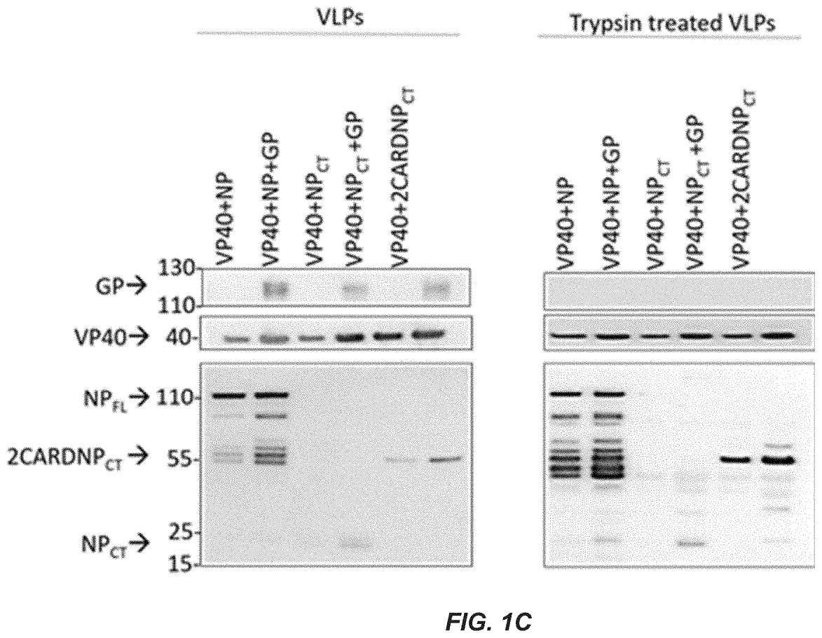

To generate Ebola VLPs (eVLPs) capable of triggering a robust antiviral response, a construct containing the two CARD domains of RIG-I (2CARD), which is capable of inducing a robust IFN response upon expression in mammalian cells, was appended to NP_{CT}, a region sufficient for interaction with VP40 and incorporation into eVLPs. A Ser-Gly linker was placed between NP_{CT} and RIG-I 2CARD (FIG. 1A). For comparison, constructs that expressed full-length NP (NP_{FL}) and NP_{CT} without the 2CARD fusion were generated. Each construct was hemagglutinin (HA)-tagged (FIG. 1A). To generate eVLPs, each construct was transfected into HEK293T cells with plasmids that express Flag-tagged VP40 and the indicated NP constructs without or with untagged GP. 48 hours post-transfection, cell supernatants were harvested and eVLPs pelleted through a 20% sucrose cushion. eVLPs were resuspended and protein concentrations determined. Endotoxin levels for all VLPs were less than 0.08 IU (endotoxin units) per microgram of total protein. By transmission electron microscopy, all eVLPs displayed the filamentous, curved morphology characteristic of authentic EBOV, and immunogold staining revealed GP spikes on the virion surface (FIG. 1B). Western blots of equivalent amounts of purified eVLPs either left untreated or treated with trypsin to degrade protein not protected within the eVLP membrane demonstrated the presence of each of the expressed viral proteins and incorporation into eVLPs of the NP_{CT} and 2CARD-NP_{CT} proteins. As expected, GP was incorporated into eVLPs when expressed and detection of the surface-exposed GP was lost upon trypsin treatment (FIG. 1C).

### 2CARD-NP_{CT} Induces an IFN Response in HEK293T Cells

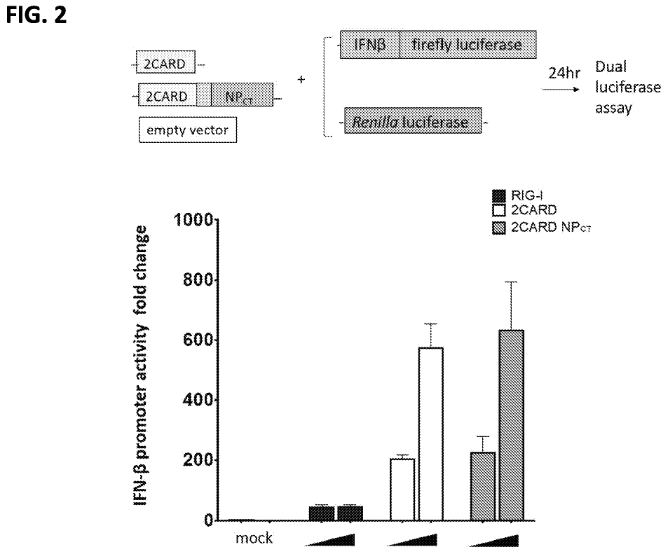

To investigate the capacity of the RIG-I 2CARD domain to induce a type I IFN response when fused to the C-terminus of Ebola virus (EBOV) NP, 293T cells were co-transfected with constructs encoding full-length RIG-I, the 2CARD domain alone, the 2CARD-NP_{CT} fusion or control empty pCAGGS vector and an IFNβ-firefly luciferase reporter plasmid. A constitutively-expressed Renilla luciferase reporter plasmid was used as an internal control. Twenty-four hours post-transfection, cell lysates were analyzed using a Dual Luciferase Assay (Promega, Madison, Wis.). As expected, full-length RIG-I did not activate the IFNβ promoter (because no activator, such as RIG-I Activating virus infection or dsRNA was added), whereas the RIG-I 2CARD construct, which lacks the regulatory RIG-I helicase domain activated the IFNβ promoter. The 2CARD-NP_{CT} construct also exhibited IFNβ-inducing capacity (FIG. 2).

2CARD VLPs Activate Enhanced IFN and Cytokine Responses in BMDCs and Stimulate Robust Antibody Responses after a Single Immunization.

To test the hypothesis that introduction of the 2CARD domain into eVLPs will enhance immune stimulating capacity, mouse bone marrow-derived DCs (BMDC) were infected with

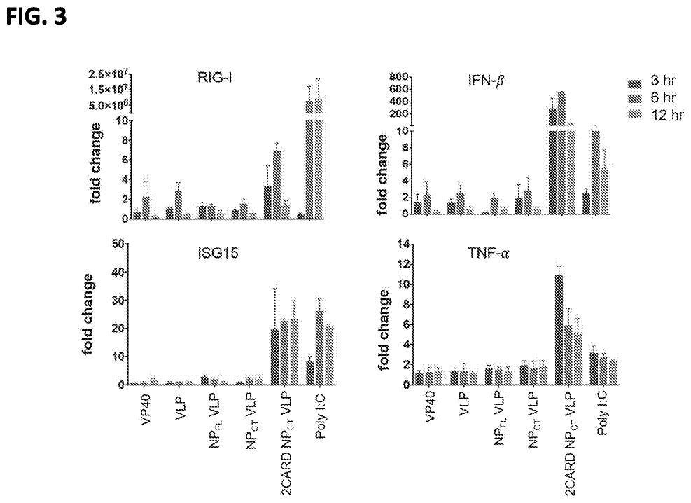

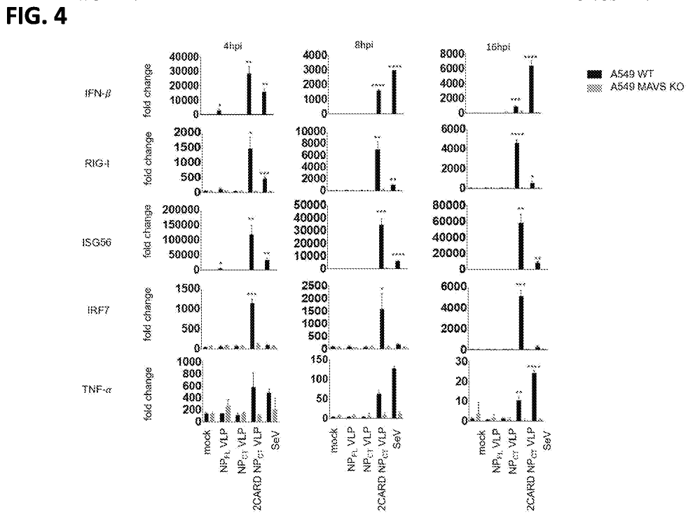

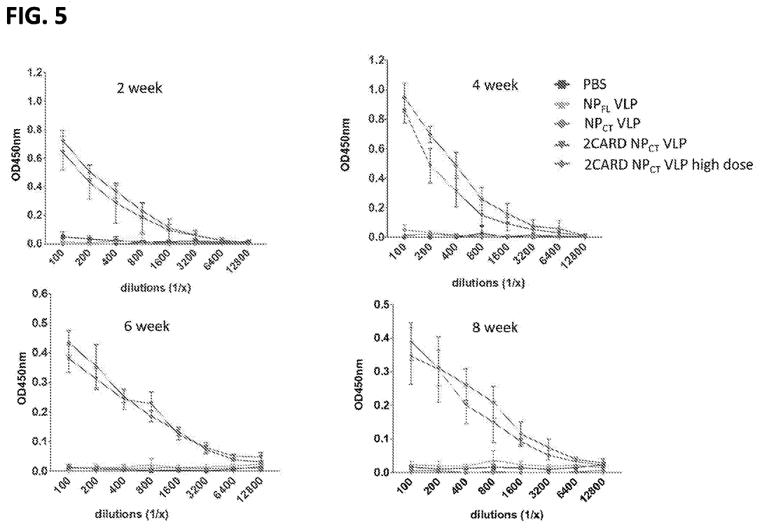

constructs expressing VP40 alone, VP40+GP, VP40+GP+NP_{FL}, VP40+GP+NP_{CT}, or VP40+GP+2CARD-NP_{CT}. As a positive control, the DCs were transfected with polyI:C, an IFN inducer (FIG. 3A). At 3, 6 and 12 hr post-infection, mRNA was isolated and quantitative RT-PCR performed. The superior capacity, relative to the other constructs, of eVLPs containing the 2CARD-NP_{CT} fusion to induce expression of IFN-β, IFN-stimulated genes RIG-I and ISG15 and cytokine TNF-α was demonstrated (FIG. 3A). Because the eVLPs were produced in human cells and tested in mice, the induced expression is not due to contaminating IFN in the eVLPs, as human IFN does not signal in mice. Further, C57BL/6 mice were immunized with a single intraperitoneal (IP) dose of 10 μg of VP40+GP+NP_{FL}, VP40+GP+NP_{CT}, VP40+GP+2CARD-NP_{CT} or a high dose (25 μg) of 2CARD eVLP; PBS served as a negative control. As expected VP40+GP+NP_{FL} eVLPs, after a single IP dose, elicited little to no detectable anti-GP antibody (FIG. 3B). In contrast either 10 μg or 25 μg of the 2CARD eVLPs gave a robust antibody response (FIG. 2B), demonstrating that the IFN inducing domain can enhance immunogenicity. These data demonstrate that enhanced VLPs, for example, EBOV VLPs that incorporate the IFN-inducing 2CARD domain through fusion to NP_{CT}, can be made.

These data demonstrate that enhanced EBOV VLPs that incorporate the IFN-inducing 2CARD domain through fusion to NP_{CT} can be made.

Mapping Regions for RIG-IN Incorporation into VLP

For the studies described above, NP₆₀₁₋₇₃₉ was initially fused to RIG-I 2CARD (FIG. 1). Structural studies suggest that NP residues 645-739 form a distinct, folded C-terminal domain (CTD). Therefore, a series of 10 amino acid deletions, from 601 towards 645, are constructed to determine whether the region of NP necessary for interaction with VP40 and incorporation into VLPs can be shortened, as assessed by coimmunoprecipitation (coIP). Each NP construct is HA-tagged at its N-terminus and Flag-tagged VP40 is used in all samples. The results are assessed by quantitative Western blot to determine minimal regions needed for efficient interaction with VP40, efficient incorporation into VLPs and for the capacity to enhance VLP release. Controls include no NP, full-length NP and the Flag-tagged NP_{CT} lacking the RIG-IN fusion. Constructs are assessed in terms of interaction with VP40, incorporation into VLP, IFN inducing capacity of the fusions upon expression in 293T cells and IFN induction in the context of VLPs, as described above.

Determine Whether Larger Fragments of NP can be Fused to RIG-IN and Still be Efficiently Incorporated into VLPs

Although NP₆₀₁₋₇₃₉ is sufficient to mediate incorporation of the RIG-IN fusion into VLPs, it is not clear if additional NP sequences might be either beneficial or detrimental. From a vaccine development perspective, additional NP sequences could prove beneficial, given that NP possesses dominant CD4+ and CD8+ T cell epitopes recognized during human infection with Ebola virus. Therefore, NP constructs with increasing amounts of N-terminal sequence (i.e. 601-739, 551-739, 501-739, 451-739) with or without RIG-IN (but each possessing an N-terminal HA tag) are compared for interaction with VP40, incorporation into VLP, IFN inducing capacity of the fusions upon expression in 293T cells and IFN induction in the context of VLPs, as described above.

### Compare Enhanced VLPs Using RIG-IN Versus TRIF as an IFN-Inducing Domain

RIG-IN is chosen as an IFN-inducing module because it represents the N-terminus of a protein that could be fused to the C-terminus of NP. However, it is not certain that RIG-IN is the optimal approach. For comparison, the TLR3 adaptor protein TRIF is fused to NP_{CT}, with a Ser-Gly linker between the two fusion partners. VP40 interaction, VLP incorporation and IFN inducing capacity of the fusions is assessed as above.

Assess Strategies to Produce Enhanced VLPs with Different Filovirus GPs and NP Sequences

EBOV, Sudan virus (SUDV), Bundibugyo virus (BDBV) and Marburg virus (MARV) are the filoviruses for which vaccines are most needed. Whether the 2CARD-NP_{CT} eVLP platform efficiently incorporate SUDV, BDBV and MARV GPs is examined. VLPs are produced and GP incorporation of the gradient purified VLPs is assessed by SDS-PAGE and Coomassie Blue staining, as described in Martinez et al. Virology 264(2): 342-54 (2007)). In addition to using untagged GP constructs, a version with a HA-tag on the cytoplasmic tail of these type I transmembrane proteins is made to compare GP incorporation by anti-Flag Western blot. If incorporation is comparable to that of EBOV GP, these are compared to the EBOV 2CARD-NP_{CT} eVLPs in the assays described above.

To match NP sequences for the virus being targeted, whether the 2CARD-NP_{CT} fusions can be swapped while retaining the EBOV VP40 is determined. EBOV, SUDV and BDBV NP constructs are likely to work with EBOV VP40 because the CTD of NPs from members of the Ebolavirus genus are well-conserved. Because the MARV NP CTD is divergent, a completely MARV-based system (2CARD-NP_{CT}+VP40+GP vs NP, VP40, GP MARV VLPs) is also evaluated. All of these constructs are assessed as described above.

### Stimulating Dendritic Cells and Eliciting Immune Responses In Vivo

As a measure of the potential for the enhanced VLPs described herein (for, example, VLPs comprising an IFN-inducing 2CARD domain) to stimulate human innate and adaptive immune responses, their impact on human DC maturation is examined. By delivering a robust IFN-inducing domain into the cytoplasm, the VLPs described herein likely can induce a more robust stimulation of DCs and more closely mimic virus infection, triggering robust DC maturation. To verify this, immature human DCs from buffy coats of anonymous human blood donors are prepared. Primary human immature DCs are mock treated or treated with standard eVLPs, enhanced eVLPs or infected with Sendai virus (strain Cantell), a potent activator of RIG-I signaling, IFN production and DC maturation. Measurements of DC responses are performed as described in Yen et al. J. Virol. 88(21): 12500-10 (2014); and Yen et al. J. Virol. 90(10): 5108-18 (2016). A Nanostring instrument and the human nCounter Myeloid Innate Immunity panel, which measures expression of 770 genes in 19 different pathways and processes across 7 different myeloid cell types are used to measure gene expression. Gene expression measurements are validated with select measurements of secreted proteins using cytometric bead assay (BD Pharmingen, Franklin Lake, N.J.) to measure inflammatory cytokines and chemokines including IL-6, IL1β, IL-12p40, TNF, RANTES and MIP-1α, because secretion of these was seen in prior eVLP assays. Release of IFNβ and IFNα are also measured by ELISA (PBL Assay Science, Piscataway, N.J.). Upregulation of MHC class I and class II and other cell surface markers of DC maturation (CD40, CD80 and CD86) is also assessed. Additionally, the capacity of the stimuli to promote DC-induced stimulation of allogeneic T cell responses is assessed.

Comparison of Adaptive Immune Responses to Enhanced eVLPs with Adaptive Immune Responses Elicited by Standard eVLPs

Three immunizations with standard, unadjuvanted eVLPs were able to confer protection of BALB/c mice and elicit robust antibody responses. Antibody and T cell responses of 2CARD-NP_{CT} eVLPs and three additional constructs exhibiting a range of IFN-inducing capacities to standard eVLPs (NP+VP40+GP) after one, two and three immunizations of BALB/c mice are compared. The goal is to identify an enhanced eVLP preparation that achieves, in a single immunization, antibody titers comparable to three doses of standard eVLPs. Immunizations are 21 days apart. 5 animals are used per group for longitudinal antibody studies (with bleeds before each immunization) with 3 animals per group for each T cell assay time point.

### ELISA Assays to Measure Anti-GP Antibody Responses

ELISA assays for anti-GP antibodies are performed as in FIG. 3B. ELISA plates are coated with recombinant EBOV GP at 1 μg/mL (rGPd™, IBT Bioservices, Rockville, Md.) in phosphate-buffered saline (PBS). Sera are diluted two-fold starting at 1:100, and horseradish peroxidase (HRP) conjugated goat anti-mouse IgG antibody (EMD Millipore, Burlington Mass.) serve as a secondary antibody. ELISAs are developed using SigmaFast OPD (Sigma, St, Louis, Mo.) and measured on a PerkinElmer EnVison (Waltham, Mass.) plate reader at OD of 450 nm. The absorbance cut-offs are defined as the background+0.1 O.D.

### Neutralization Assays

Vesicular stomatitis viruses (VSV) in which the VSV glycoprotein (G) is replaced by the EBOV GP serves as a useful biosafety level 2 surrogate for EBOV neutralization assays, with the VSV-GP-based assay highly correlating with neutralization performed with authentic EBOV (Regules et al. N. Engl. J. Med. 376(4): 330-41 (2017); and Konduru et al. J. Virol. Methods 254:1-7 (2018)). A VSV-GP that also encodes, as an extra viral gene, green fluorescence protein (GFP) is also used. This VSV-GFP-GP virus is also used for neutralization assays, as described in Duehr et al. J. Virol. 91(16) (2017). Pre-bleed sera are included as negative controls. Neutralizing anti-GP mouse mAb 1F8 are used as a positive control.

### T Cell Responses

A peptide restimulation assay is used to evaluate CD4 and CD8 T cell responses at 2 weeks post-VLP vaccination, as assessed by interferon (IFN)-γ production. The peptides are Zaire Ebola virus BALB/c T cell epitopes for GP (LYDRLASTV (G₁₆₁₋₁₆₉)) (SEQ ID NO: 5) and VP40, for example, (YFTFDLTALK (VP₁₇₁₋₁₈₀) (SEQ ID NO: 6) and PEYMEAIYPVRSNST (VP₁₁₋₂₅)) (SEQ ID NO: 7). For NP, the reported mouse epitopes lie outside the NP_{CT} domain used in the studies described above. Therefore, these epitopes are tested in studies evaluating 2CARD fusions to VP40 rather than NP. Splenocytes isolated at 2 weeks post-vaccination are cultured for 5 h in RPMI 1640 with or without 1 μM synthetic peptide and 10 μg/ml brefeldin A is added for the final 2 h. The cells are blocked with mAbs to FcRIII/II and stained with anti-CD8 CyChrome, or anti-CD4 CyChrome (BD Pharmingen) with brefeldin A. Following fixation with 1% formaldehyde, the cells are permeabilized with 0.5% saponin, stained with anti-IFN-γ PE (BD Pharmingen), and analyzed by flow cytometry on a SONY SH800 FACS (Champaign, Ill.). A >2-fold increase in the frequency of IFN-γ-positive cells, as compared to irrelevant peptide or no peptide, is considered positive.

### Proliferation and Cytokine Production

Splenocytes obtained at 2 weeks post-vaccination are assayed for proliferation by culturing in RPMI 1640 medium in the absence or presence of 10 μg/ml eVLP. After 5 days in culture, aliquots of culture supernatants are removed for cytokine analysis and 1 μCi of [³H]thymidine is added to each well, with ³H incorporation determined after 18 h incubation. The levels of cytokines in the supernatants of the restimulated cells are determined using a cytometric bead assay (BD Pharmingen) to detect IL-2, IL-4, IL-5, IL-6, IL-10, IL-12p70, IFN-γ, and TNF-α.

### T Cell Enzyme-Linked Immunosorbent Spot-Forming Cell Assay (ELISPOT) Assays.

T cell memory responses are assessed by using a “cultured IFN-γ ELISPOT” approach, which reveals potential IFN-γ-secreting T cell capacity within a lymphocyte population (Todryk et al., 128(1): 83-91 (2009). Lymphocytes isolated from spleen and mediastinal LN are cultured in RPMI with peptides consisting of CD8 and CD4 epitopes of GP and VP40 as detailed in the peptide restimulation assay above, as well as full length recombinant GP protein (IBT Bioservices). Cultures are supplemented with 100 U/ml IL-2 on days 3 and 7. On day 9, the cells are washed and rested overnight, before performing an IFN-γ ELISPOT assay (Mabtech, Cincinnati, Ohio). Medium-only, irrelevant peptide and phytohaemagglutinin controls are included. The results are compared with a standard ex vivo IFN-γ ELISPOT run in parallel, where culture with antigen is only overnight rather than over 9 days. The ELISPOT assays are conducted at 4 days, 4 weeks, 4 months post-vaccination, to examine durability of responses.

### B Cell Memory Responses

Memory B cell responses are studied using a modification of the limiting dilution assay (Ndungu et al. PLoS Pathog. 5(12):e1000690 (2009); and Slifka et al. J. Immunol. Methods 199(1): 37-46 (1996)). Replicates of three-fold dilutions of cell suspensions of spleen and bone marrow are cultured for 6 days in Iscove's medium containing R595 lipopolysaccharide (Sigma), 1×10⁶ irradiated naive splenocytes, and 1/10th volume supernatant from concanavalin A-stimulated splenocytes. Cells are harvested and transferred to 96-well Multi-screen HA Nitrocellulose filtration plates (Millipore) coated with 50 μl of 2.5 μg/ml recombinant GP diluted in PBS and an ex-vivo ELISpot assay for GP-specific antibody-secreting cells performed according to the MabTech (Cincinnati, Ohio) ELISpot protocol.

### Prime-Boost Immunization Strategy

Mice are vaccinated by the IP route with 1 or 10 μg of standard or enhanced eVLPs. In a prior study using a prime-boost approach with standard eVLPs it was found that a 10 μg dose was protective after 2 boosts but 1 μg was not (protection with fewer immunizations was not reported). A prime-boost with a lower dose of enhanced eVLPs is selected for protection. For these studies, control mice receive PBS as a vaccine. The control PBS group and the VLP immunized groups consist of 5 or 10 mice, respectively. Priming occurs on day 0 and boosting on day 21. Mice are challenged 6 weeks post vaccination by IP injection with 1,000 plaque forming units (a 100% lethal dose) of mouse-adapted EBOV (maEBOV). Post-challenge, mice are observed twice daily for illness and serum viremia on day 4, the typical peak day of viremia. Viremia is assessed both by performing quantitative RT-PCR for viral genome numbers and by plaque assay on Vero E6 cells. Mice are weighed daily following challenge and observed for clinical symptoms according to an approved scoring sheet (ruffled fur, slowing activity, loss of body conditions, labored breathing, hunched posture, bleeding, paralysis). Mice are monitored for up to 28 days post-challenge to look for signs of delayed illness. For all animals that reach humane endpoints, blood is obtained and liver, spleen, lung, kidney and brain are harvested. For blood, viral RNA levels are assayed by quantitative RT-PCR, and viral titers are measured by plaque assay. Other tissues are assessed for viral antigen by immunohistochemistry, for viral RNA by quantitative RT-PCR and for viral titers by plaque assay. ELISAs to measure antibodies to virions and to GP are performed on sera obtained prior to challenge, obtained on day 4 post-challenge and in survivors 28 days post-challenge.

Testing Optimized, Enhanced eVLPs for Protection after a Single Immunization

A single dose of enhanced eVLPs sufficient to elicit anti-GP antibody responses that is comparable to three immunizations with standard eVLPs is determined. These experiments follow the procedures described above, except that challenge will occur on day 28 post-vaccination.

Incorporation of a Bacterial Antigen into Interferon-Inducing eVLPs

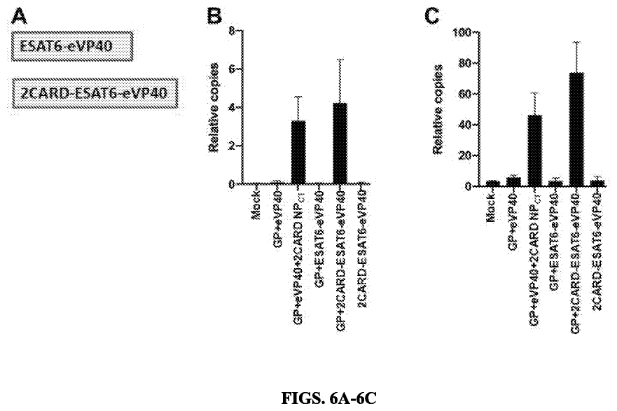

Mycobacterium tuberculosis (Mtb) is the causative agent of tuberculosis. A vaccine effective against pulmonary tuberculosis is needed. The use of an Ebola VLP vaccine approach described herein to deliver an Mtb antigen in such a form that the immune enhancing, interferon-inducing 2CARD domain will also be present in the VLPs, was studied. For this experiment, the Mtb protein, ESAT6, was chosen. as this is a known antigen that has been used on other vaccine platforms. The innovation with this approach is the use of an enhanced 2CARD VLP as an Mtb vaccine. VLPs were generated by fusing ESAT6 to EBOV VP40 (ESAT6-VP40) (FIG. 6A) and a corresponding 2CARD-ESAT6-eVP40. To test whether these VLPs can stimulate innate immune responses upon infection of cells, the following experiment was performed.

VLPs were produced by transfecting the following plasmids into HEK293T cells: GP+eVP40; GP+eVP40+2CARD-NP_{CT}; GP+ESAT6-eVP40; GP+2CARD-ESAT6-eVP40; 2CARD-ESAT6-eVP40. After harvesting the VLPs, the VLPs were added to HEK293T cells. As a control, the HEK293T cells were mock treated. Activation of innate immune responses were then determined by measuring levels of mRNAs to interferon β(IFNβ) and the IFNβ-induced gene RIG-I. These mRNA levels were normalized to β-actin mRNA levels and reported as relative copies of the IFNβ or RIG-I mRNAs (FIG. 6A and FIG. 6B, respectively). As seen in prior experiments, mock treatment did not induce IFNβ or RIG-I mRNA expression. Similarly, GP+eVP40+2CARD-NP_{CT} VLPs induced IFNβ and RIG-I mRNA expression, as seen in prior experiments. GP+ESAT6-eVP40 VLPs did not induce IFNβ and RIG-I mRNA expression, consistent with the lack of the IFNβ-inducing 2CARD domain. However, GP+2CARD-ESAT6-eVP40 did induce these genes, as expected, given the presence of 2CARD. When 2CARD-ESAT6-eVP40 was expressed without GP, the resulting VLPs did not induce expression of IFNβ and RIG-I mRNA. This is consistent with the model that GP must be present on the VLPs to allow delivery of the 2CARD-containing protein into cells to induce IFNβ and RIG-I mRNA expression.

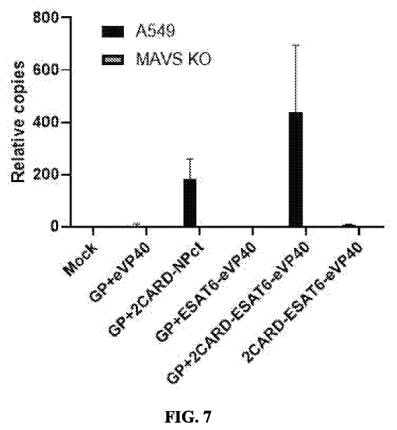

When these VLPs are used to infect a wildtype version of the A549 cell line, the 2CARD-ESAT6-eVP40 VLPs trigger a type I interferon (IFN) response that is as strong or stronger than the control 2CARD-NPct+eVP40 eVLPs (which do not contain Mtb antigen) (FIG. 7). The latter are the VLPs described above that provided evidence for using a 2CARD-packaging eVLPs to induce a very robust adaptive immune response. Importantly, the IFN response is eliminated when A549 cells deleted for the cellular signaling molecule MAVS are infected (FIG. 7). 2CARD-induced IFN responses are expected to require MAVS as a downstream signaling molecule to trigger IFN responses. Because the IFN response induced by 2CARD-containing eVLPs is MAVS-dependent, these results show that the IFN induction proceeds by the expected mechanism, even in the case of the ESAT6 containing eVLPs.

What is claimed is:

1. A virus-like particle (VLP) comprising:
a. a surface protein;
b. a matrix protein;
c. a polypeptide that enhances an immune response, wherein the polypeptide is a polypeptide adjuvant and wherein the polypeptide is linked to the surface protein, the matrix protein, or an intra-VLP protein.

2. The VLP of claim 1, wherein the surface protein induces a B-cell mediated immune response.

3. The VLP of claim 1 or 2, wherein the intra-VLP protein or a fragment thereof induces a T cell-mediated immune response.

4. The VLP of any one of claims 1-3, wherein the polypeptide adjuvant enhances a B-cell mediated immune response, a T cell-mediated immune response, or both a B-cell mediated immune response and a T cell-mediated immune response.

5. The VLP of any one of claims 1-4, wherein the polypeptide adjuvant comprises one or more signaling domains.

6. The VLP of claim 5, wherein the one or more signaling domains comprise caspase activation and recruitment domains (CARDs).

7. The VLP of claim 5 or 6, wherein the polypeptide induces a Type I interferon immune response.

8. The VLP of any one of claims 1-7, wherein the immune response is an anti-pathogenic immune response.

9. The VLP of claim 7, wherein the anti-pathogenic immune response is an antiviral immune response, an antibacterial immune response, an antifungal immune response, or an antiparasitic immune response.

10. The VLP of any one of claims 1-7, wherein the immune response is an anticancer immune response

11. The VLP of any one of claims 1-9, wherein the surface protein comprises a bacterial protein.

12. The VLP of any one of claims 1-9, wherein the matrix protein or the intra-VLP protein comprises a bacterial protein.

13. The VLP of claim 11 or 12, wherein the bacterial protein is a mycobacterial protein.

14. The VLP of any one of claim 1-7 or 10, wherein the surface protein comprises a cancer antigen.

15. The VLP of any one of claims 1-14, wherein the surface protein comprises a viral surface protein.

16. The VLP of any one of claims 1-15, wherein the matrix protein comprises a viral matrix protein.

17. The VLP of any one of claims 1-16, wherein the intra-VLP protein or a fragment thereof comprises a viral nucleoprotein (NP) or a fragment thereof.

18. The VLP of claim 17, wherein the viral NP fragment is a C-terminal fragment of a viral NP.

19. The VLP of claim 17 or 18, wherein the viral surface protein, the viral matrix protein and the viral NP or a fragment thereof are from the same virus.

20. The VLP of claim 19, wherein the virus is selected from the group consisting of a filovirus, an arenavirus, a paramyxovirus, a pneumovirus, and an influenza virus.

21. The VLP of claim 17 or 18, wherein the viral surface protein, the viral matrix protein and the viral NP or a fragment thereof are from different strains of the same virus or different viruses.

22. The VLP of claim 19, wherein the different strains or different viruses are selected from the group consisting of a filovirus, an arenavirus, a paramyxovirus, a pneumovirus, and an influenza virus.

23. The VLP of any one of claims 5-22, wherein the intra-VLP protein or a fragment thereof is linked to a polypeptide comprising two signaling domains.

24. The VLP of any one of claims 5-23, wherein at least one signaling domain is a CARD domain.

25. The VLP of claim 24, wherein the at least one signaling domain is a CARD domain from a RIG-I-like receptor.

26. The VLP of claim 25, wherein the RIG-I-like receptor is retinoic acid-inducible gene-I (RIG-I).

27. The VLP of claim 25, wherein the RIG-I-like receptor is Melanoma Differentiation-Associated protein 5 (MDA5).

28. The VLP of any one of claims 16-27, wherein the viral matrix protein is an Ebola virus VP40 matrix protein.

29. The VLP of claim 28, wherein the viral surface protein is an Ebola virus glycoprotein (GP).

30. The VLP of claim 29, wherein the viral NP is an Ebola virus NP or a fragment thereof.

31. The VLP of claim 21, wherein the viral surface protein and the viral NP or a fragment thereof are from a different strain or a different virus.

32. The VLP of any one of claims 15-27, wherein the viral surface protein is a Lassa virus GPC.

33. The VLP of claim 32, wherein the viral matrix protein is a Lassa virus matrix protein Z.

34. The VLP of claim 32 or 33, wherein the viral nucleoprotein is a Lassa virus NP or a fragment thereof.

35. The VLP of any one of claims 15-27, wherein the viral surface protein is an influenza virus glycoprotein.

36. The VLP of claim 35, wherein the influenza virus glycoprotein is an influenza hemagglutinin (HA).

37. The VLP of claim 35 or 36, wherein the viral matrix protein is an influenza M1 protein.

38. The VLP of claim 37, wherein the viral nucleoprotein is an influenza virus NP or a fragment thereof.

39. The VLP of any one of claims 15-27, wherein the viral surface protein is a henipavirus glycoprotein.

40. The VLP of claim 39, wherein the henipavirus glycoprotein is a Nipah virus F protein or a Nipah virus G protein.

41. The VLP of claim 39 or 40, wherein the viral matrix protein is a Nipah virus matrix (M) protein.

42. The VLP of claim 41, wherein the viral nucleoprotein is Nipah virus NP or a fragment thereof.

43. An isolated host cell that expresses the VLP of any one of claims 1-42.

44. The isolated host cell of claim 43, wherein the host cell is a mammalian cell.

45. The isolated host cell of claim 44, wherein the host cell is an insect cell.

46. An immunogenic composition comprising the VLP of any one of claims 1-42 and a pharmaceutically acceptable carrier.

47. A method of making the immunogenic composition of claim 46 comprising:
(a) expressing in a host cell at least one VLP of any one of claims 1-42;
(b) growing the host cell under conditions which allow the formation of VLPs;
(c) purifying the VLPs, and
(d) preparing the immunogenic composition with the purified VLPs.

48. The method of claim 47, wherein the host cell is transfected or infected with one or more recombinant constructs encoding (a) the surface protein; (b) the matrix protein, (c) the intra-VLP protein or a fragment thereof; and (d) a polypeptide that enhances an immune response, wherein the polypeptide is linked to the surface protein, the matrix protein or the intra-VLP protein or a fragment thereof.

49. An immunogenic composition comprising at least one VLP produced by the method of claim 47 or 48.

50. A method of stimulating an immune response in a subject comprising administering to the subject an effective amount of the immunogenic composition of claim 49.

51. The method of claim 50, wherein the immunogenic composition is administered to the subject as a single dose.

52. The method of claim 50 or 51, wherein the immunogenic composition enhances an immune response in the subject.

53. The method of any one of claims 50-52, wherein the immune response is an immune response against a pathogen in the subject.

54. The method of any one of claims 50-52, wherein the immune response is an anticancer immune response.

55. The method of claim 53, wherein the pathogen is a virus, a bacterium, a fungus, or a parasite.

56. The method of claim 55, wherein the virus is selected from the group consisting of a filovirus, an arenavirus, a henipavirus, a pneumovirus, and an influenza virus.

57. The method of claim 56, wherein the virus is selected from the group consisting of Ebola virus, Nipah virus, Lassa virus and influenza virus.

58. The method of claim 53, wherein the pathogen is a bacterium.

59. The method of claim 58, wherein the bacterium is a mycobacterium.
